(19) United States
(12) Patent Application Publication
(10) Pub. No.: US 2007/0065487 A1
(43) Pub. Date: Mar. 22, 2007

(54) Trans-lutein xantophyll ester of high purity and high bioavailability in micellar solution and a process for the preparation thereof

(75) Inventor: Alejandro Ornelas Cravioto, Guanajuato (MX)

Correspondence Address: OBLON, SPIVAK, MCCLELLAND, MAIER & NEUSTADT, P.C., 1940 DUKE STREET, ALEXANDRIA, VA, 22314, US

(73) Assignee: BIO LUT S.A. de C.V., San Antonio, TX (US)

(21) Appl. No.: 11/230,601

(22) Filed: Sep. 21, 2005

(51) Int. Cl. A61K 31/22; A61K 47/00

(52) U.S. Cl. 424/439; 514/548

(57) ABSTRACT

A colloidal crystalline solution comprising a xanthophyll ester having a high trans-lutein content and at least one oil is provided. The solution may be prepared by dissolving the xanthophyll ester in the oil. Methods of using the solution are also provided.

### BACKGROUND OF THE INVENTION

1. Field of the Invention

The present invention relates to colloidal crystalline solutions containing a xanthophyll ester having a high trans-lutein content and an oil. The present invention also includes methods of making such solutions and uses thereof.

2. Description of the Background

Xanthophyll esters belong to a group of natural compounds known as carotenoids and are widely distributed in nature. Xanthophyll esters are mainly fatty acid esters [e.g., palmitate and myristate esters; J. of Food Sci., 51 (4) 1093 (1986)] of carotenoids such as lutein and zeaxanthin. The marigold flower (tagetes erecta) is the richest source of trans-lutein esters found in nature. Xanthophyll esters are generally found in nature as the trans-xanthophyll isomer. Recent scientific research has shown that marigold extracts may be used as nutritional supplements. It has been found that trans-lutein esters are specially beneficial in the treatment and prevention of some types of cancer and of a condition known as age-related degeneration of the macula of the human eye (MAD), (“The effect of dietary lutein and growth of mammary tumor BALB /c Mice,” The FASEB Journal 11 2586 (1977); International Journal of Cancer 63 18-23 ( 1995 )).

The xanthophylls in particular have been shown to possess strong antioxidant activities and may be useful in protecting humans from certain diseases. For example trans-lutein and trans-zeaxanthin are orange to reddish pigments that have been inversely correlated with reduction of risk for macular degeneration (Seddon et al., 1994, J. Amner. Med. Assos. 272 (18), 1413- 1420).

Several indicative studies have demonstrated that lutein esters increase the macular pigment density and the bioavailability even more effectively than free lutein (Herbst S, Bowen P, Hussein E, Bums J., “Evaluation of the Bioavailability of lutein (L) and Lutein ester (LD) in humans,” The FASEB Journal, 1997; 11:2587 (Abstr.).

When lutein esters are used for human consumption, it is preferable to have the highest possible concentration as well as the highest purity by HPLC once the ester is saponified. When consumption of trans-lutein esters is recommended for certain diseases related to macular degeneration or just for the natural antioxidant properties, most of the literature recommends the use of suspensions of lutein esters in concentrations from 4-50% by weight based on the total weight of the composition (U.S. Pat. No. 6,313,169).

There remains a need for novel compositions containing lutein esters.

### SUMMARY OF THE INVENTION

It is an object of the present invention to provide solutions of xanthophyll esters.

It is another object of the present invention to provide methods of making such solutions.

The objects of the invention, and others, may be accomplished with a colloidal crystalline solution comprising a xanthophyll ester having a high trans-lutein content and at least one oil.

The objects of the invention may also be accomplished with a method of preparing the solution described above, comprising dissolving the xanthophyll ester in the oil.

### DETAILED DESCRIPTION OF THE INVENTION

The present invention provides a colloidal crystalline solution comprising a xanthophyll ester having a high trans-lutein content and at least one oil.

In a preferred embodiment the solution is substantially free of an emulsifier. That is, the solution is substantially free of any additional substances which emulsify the xanthophyll ester in the oil.

In one embodiment, the solution is a micellar solution. In another embodiment, the solution is substantially free of suspended and/or precipitated material.

In a preferred embodiment of the invention, the xanthophyll ester is composed of at least about 94% of trans-lutein. In a more preferred embodiment, the xanthophyll ester is composed of about 94-96% of trans-lutein. In a particularly preferred embodiment, the xanthophyll ester is composed of about 94-96% of trans-lutein, 0.1-1.0% of cis-lutein and 3-5% of trans-zeaxanthin.

In one embodiment, the oil in the solution of the invention is an edible oil. In a preferred embodiment, the oil is a vegetable oil. In a particularly preferred embodiment, the oil is coconut oil, corn oil, palm oil, peanut oil, olive oil, sesame oil, soy bean oil and mixtures thereof.

In a preferred embodiment of the invention, the solution contains 2 to 15 g/kg of the xanthophyll ester. That range includes all specific values and subranges therebetween, such as 5, 8, 10 and 12 g/kg. In a more preferred embodiment, the solution contains 2 to 15 g/kg of the xanthophyll ester and wherein the xanthophyll ester is composed of about 94-96% of trans-lutein, 0.1-1.0% of cis-lutein and 3-5% of trans-zeaxanthin.

In one embodiment of the invention, the solution consists essentially of the xanthophyll ester and the oil. In another embodiment, the solution consists of the xanthophyll ester and the oil.

In another embodiment of the invention, the solution further comprises at least one stabilizer. In a preferred embodiment the stabilizer is non-toxic. The stabilizer may be an antioxidant.

In another embodiment, the solution is substantially free of trans-lutein.

The present invention also provides a method of preparing the solution, comprising dissolving the xanthophyll ester in the oil. In one embodiment, the xanthophyll ester is dissolved in the oil at a temperature of about 5-95° C. That range includes all specific values and subranges therebetween, such as 10, 15, 20, 25, 30, 35, 50, 45, 50, 55, 60, 65, 70, 75, 80, 85 and 90° C. Preferably, the oil is hot, e.g., above a temperature of about 40° C.

In a preferred embodiment, the method further comprises removing solids from the xanthophyll ester dissolved in the oil. In one embodiment, the solids are removed by filtration. In another embodiment, the solids are removed by centrifugation or sedimentation. As a result, the solution is substantially free of suspended and/or precipitated material.

In one embodiment, the xanthophyll content of the xanthophyll ester to be dissolved in the oil is from 100 to 550 grams of xanthophylls/kilo. That range includes all specific values and subranges therebetween, such as 150, 200, 250, 300, 350, 400, 450 and 500 grams of xanthophylls/kilo. The xanthophyll ester to be dissolved in the oil may be obtained as described in U.S. patent application Ser. No. 10/986,049, incorporated herein by reference.

The product provided by the present invention is suitable for human consumption and can be used as an additive in edible compositions. The product of the present invention may also be incorporated into compositions which are intended for topical use, i.e., application to the skin. Such a composition may be a food or beverage product. In preferred embodiments, the composition of the present invention can be incorporated into health foods and nutritive supplements, e.g., as a colorant. In addition, the trans-lutein esters have been studied with respect to preventing eye diseases such as cataracts and aging macular degeneration; they also have been studied with respect to the treatment of certain diseases like cancer and cardiovascular ailments. Accordingly, the composition of the present invention can be used to treat such conditions. Humans are the preferred subjects for such treatments. These uses are well-known generally are described in, for example, U.S. Pat. Nos. 6,787,147; 6,787,151; 6,716,451; 6,686,340; and 6,660,297, all incorporated herein by reference.

### EXAMPLES

### Example 1

Dissolve 4 g a free flowing dry xanthophyll ester with a high trans-lutein ester content having a concentration of 480 grams of xanthophylls per kilo in 96 g of soybean oil at 60° C. using a magnetic stirrer. Once the product has dissolved let the solution stand in order to reach room temperature. When the solution has reached room temperature, filter the solution through a No. 2 What man filter paper. If some precipitate appears in this first filtration repeat the operation until no precipitate appears.

The oily colloidal crystalline solution with high xanthophyll ester concentration obtained in this way contains 6.37 g/kg of total xanthophylls and a chromathographic profile by HPLC of 94-96% of trans-lutein, 0.1-1.0% of cis-lutein and 3-5% of trans-zeaxanthin.

### Example 2

Dissolve 8 g of a free flowing dry xanthophyll ester with a high trans-lutein ester having a concentration of 400 grams of xanthophylls /kilo in 96 g of soybean oil at 60° C. using a magnetic stirrer. Once the product has dissolved let the solution stand to reach room temperature. When the solution has reached room temperature, filter the solution through a No. 2 What man filter paper. If some precipitate appears in this first filtration repeat the operation until no precipitate appears.

The oily colloidal crystalline solution with high xanthophyll ester concentration obtained in this way contains 13.78 g/kg of total xanthophylls and a chromathographic profile by HPLC of 94-96% of trans-lutein, 0.1-1.0% of cis-lutein and 3-5% of trans-zeaxanthin.

### Example 3

Dissolve 12 g a free flowing dry xanthophyll ester with a high trans-lutein ester content having a concentration of 520 g of xanthophylls /kilo in 96 g of soybean oil at 60° C. using a magnetic stirrer. Once the product has dissolved let the solution stand in order to reach room temperature. When the solution has reached room temperature, filter the solution through a No. 2 Whatman filter paper. If some precipitate appears in this first filtration repeat the operation until no precipitate appears. The oily colloidal crystalline solution with high xanthophyll ester concentration obtained in this way contains 15.22 g/kg of total xanthophylls and a chromathographic profile by HPLC of 94-96% of trans-lutein, 0.1-1.0% of cis-lutein and 3-5% of trans-zeaxanthin.

1. A colloidal crystalline solution comprising a xanthophyll ester having a high trans-lutein content and at least one oil.

2. The solution of claim 1, which is substantially free of an emulsifier.

3. The solution of claim 1, wherein the xanthophyll ester is composed of at least about 94% of trans-lutein.

4. The solution of claim 1, wherein the xanthophyll ester is composed of about 94-96% of trans-lutein.

5. The solution of claim 1, wherein the xanthophyll ester is composed of about 94-96% of trans-lutein, 0.1-1.0% of cis-lutein and 3-5% of trans-zeaxanthin.

6. The solution of claim 1, wherein the oil is an edible oil.

7. The solution of claim 1, wherein the oil is a vegetable oil.

8. The solution of claim 1, wherein the oil is coconut oil, corn oil, palm oil, peanut oil, olive oil, sesame oil, soy bean oil and mixtures thereof.

9. The solution of claim 1, which is a micellar solution

10. The solution of claim 1, which is substantially free of suspended and/or precipitated material.

11. The solution of claim 1, which contains 2 to 15 g/kg of the xanthophyll ester.

12. The solution of claim 1, which contains 2 to 15 g/kg of the xanthophyll ester and wherein the xanthophyll ester is composed of about 94-96% of trans-lutein, 0.1-1.0% of cis-lutein and 3-5% of trans-zeaxanthin.

13. The solution of claim 1, which consists essentially of the xanthophyll ester and the oil.

14. The solution of claim 1, which consists of the xanthophyll ester and the oil.

15. The solution of claim 1, which further comprises at least one stabilizer.

16. The solution of claim 1, wherein the stabilizer is non-toxic.

17. The solution of claim 1, which further comprises at least one antioxidant.

18. The solution of claim 1, wherein the antioxidant is non-toxic.

19. The solution of claim 1, which is substantially free of trans-lutein.

20. A method of preparing the solution of claim 1, comprising dissolving the xanthophyll ester in the oil.

21. The method of claim 20, wherein the xanthophyll ester is dissolved in the oil at a temperature of about 5-95° C.

22. The method of claim 20, wherein the xanthophyll ester is dissolved in the oil at a temperature of about 15-95° C.

23. The method of claim 20, wherein the xanthophyll ester is dissolved in the oil at a temperature of about 15-75° C.

24. The method of claim 20, wherein the xanthophyll ester is dissolved in the oil at a temperature of about 25-75° C.

25. The method of claim 20, wherein the xanthophyll ester is dissolved in the oil at a temperature of about 80° C.

26. The method of claim 20, wherein the xanthophyll ester is dissolved in the oil at a temperature of about 60° C.

27. The method of claim 21, which further comprises removing solids from the xanthophyll ester dissolved in the oil.

28. The method of claim 27, wherein the solids are removed by filtration.

29. The method of claim 27, wherein the solids are removed by centrifugation or sedimentation.

30. The method of claim 20, wherein the solution is substantially free of suspended and/or precipitated material.

31. The method of claim 20, wherein the xanthophyll content of the xanthophyll ester is from 100 to 550 grams of xanthophylls/kilo.

32. A food or beverage composition, comprising at least one food or beverage and the solution of claim 1.

33. A method of making a food or beverage composition comprising combining at least one food or beverage and the solution of claim 1.

34. A method of making a health food or a dietary supplement, comprising incorporating the solution of claim 1 into a health food or a dietary supplement.

35. A method of making a food or beverage product, comprising incorporating the solution of claim 1 into a food or beverage product.

36. A method of treating an eye disease, comprising administering an effective amount of the solution of claim 1 to a subject in need thereof.

37. The method of claim 36, wherein the subject is a human.

38. The method of claim 36, wherein the eye disease is cataracts or aging macular degeneration.

39. A method of treating cancer, comprising administering an effective amount of the solution of claim 1 to a subject in need thereof.

40. The method of claim 39, wherein the subject is a human.

41. A method of treating a cardiovascular ailment, comprising administering an effective amount of the solution of claim 1 to a subject in need thereof.

42. The method of claim 41, wherein the subject is a human.
